(19) United States
(12) Patent Application Publication
(10) Pub. No.: US 2014/0186431 A1
(43) Pub. Date: Jul. 3, 2014

(54) COMBINED MESENCHYMAL STEM CELL TRANSPLANTATION AND TARGETED DELIVERY OF VEGF FOR TREATMENT OF MYOCARDIAL INFARCTION

(71) Applicant: Temple University - Of The Commonwealth System of Higher Education, Philadelphia, PA (US)

(72) Inventors: Bin Wang, Thornton, PA (US); Mohammad F. Kiani, Wynnewood, PA (US); Yuan Tang, Elkins Park, PA (US)

(73) Assignee: Temple University - Of The Commonwealth System of Higher Education, Philadelphia, PA (US)

(21) Appl. No.: 14/096,449

(22) Filed: Dec. 4, 2013

Related U.S. Application Data
(60) Provisional application No. 61/748,609, filed on Jan. 3, 2013.

(51) Int. Cl. A61K 35/28; A61K 38/18; A61K 9/127

(52) U.S. Cl. CPC A61K 35/28; A61K 9/127; A61K 38/1866
USPC 424/450; 424/93.7

(57) ABSTRACT

Compositions and kits useful for the treatment of myocardial infarction comprise (i) P-selectin-targeted carriers (e.g., P-selectin-targeted liposomes, quantum dots or biodegradable nanoparticles) comprising VEGF and (ii) MSCs.

### CROSS-REFERENCE TO RELATED APPLICATION

The benefit of the filing date of U.S. Provisional Patent Application No. 61/748,609, filed Jan. 3, 2013, is hereby claimed. The entire disclosure of the aforesaid application is incorporated herein by reference.

### FIELD OF THE INVENTION

The present invention relates to combined mesenchymal stem cell transplantation and targeted delivery of VEGF for treatment of myocardial infarction.

### BACKGROUND OF THE INVENTION

Myocardial infarction (MI) is the leading cause of morbidity and mortality in most countries (Dickstein et al., Prog Cardiovasc Dis. 2012; 54:362-366). MI occurs when blood supply to the heart is disrupted for a long enough time that results in cell loss and myocardium necrosis. The damaged area will eventually be replaced by scar tissue to maintain the structural integrity. However cardiac function will never restore because myocardium cannot regenerate by itself (Tiyyagura et al., Mt Sinai J Med. 2006; 73:840-851).

Current clinical treatment procedure for MI is focused on first restoring the antegrade flows and minimizing pathologic remodeling of the MI. Heart function can then be improved by administration of inotropic drugs to boost the mechanical performance of remaining cardiomyocytes (Forte et al., Stem Cell Rev. 2011; 7:1018-1030). However, these treatment modalities only ameliorate symptoms but do not recover the function of the damaged tissue.

In order to recover heart function, an MI area needs to be repaired by generating new myocardiocytes. Tissue engineering approaches employing stem cells have achieved laboratory success in this respect as stem cells can be induced to differentiate into cardiac myocytes and thus have the potential to regenerate new myocardium (Passier et al., Nature. 2008; 453:322-329; Copland, J Cardiovasc Dis Res. 2011; 2:3-13). However, attempts at rebuilding injured tissue in vivo using transplanted stem cells has yet to be successful due in part to a lack of blood supply to the MI region leading to poor viability and increased apoptosis of the transplanted cells (Scott et al., Expert Opin Drug Deliv. 2008; 5:459-470). Pro-angiogenic compounds such as vascular endothelial growth factor (VEGF) have been shown to stimulate blood vessel regrowth back into the MI area (Hughes et al., Ann Thorac Surg. 2004; 77:812-818; Freedman et al., Ann Intern Med. 2002; 136:54-71). However, systemic delivery of VEGF has very limited therapeutic effects (Scott et al., FASEB J. 2009; 23:3361-3367) and can produce many severe side effects such as the development of neoplasms, diabetic retinopathy, rheumatoid arthritis, and atherosclerosis (Inoue et al., Circulation. 1998; 98:2108-2116; Lee et al., Circulation. 2000; 102:898-901; Pettersson et al., Lab Invest. 2000; 80:99-115; Schwarz et al., J Am Coll Cardiol. 2000; 35:1323-1330; Lazarous et al., Circulation. 1996; 94:1074-1082; Epstein et al., Circulation. 2001; 104:115-119). Targeted delivery methods can concentrate VEGF at the MI site thus maximizing its therapeutic potential while minimizing its side effects.

Previously it has been reported that targeted delivery of VEGF to infracted myocardium with anti-P-selectin conjugated immunoliposomes results in a significant increase in the number of both anatomical and perfused vessels in the MI region in rats and improvements in cardiac function (Scott et al., FASEB J. 2009; 23:3361-3367).

There remains a need for a treatment that can enhance the survival of mesenchymal stem cells and their engraftment.

### SUMMARY OF THE INVENTION

Provided is a method for treating myocardial infarction comprising administering to a subject in need of such a treatment an effective amount of a composition comprising P-selectin-targeted carriers comprising VEGF and intramyocardially administering to said subject an effective amount of mesenchymal stem cells (MSCs). In some embodiments, the P-selectin-targeted carriers are P-selectin-targeted liposomes, P-selectin-targeted quantum dots or P-selectin-targeted biodegradable nanoparticles. In some embodiments the P-selectin-targeted liposomes are immunoliposomes. In some embodiments the P-selectin-targeted liposomes comprise hydrogenated soy L-α-phosphatidylcholine, cholesterol, 1,2-distearoyl-sn-glycero-3-phosphoethanolamine-N-[(polyethylene glycol)2000] and DSPE-PEG2000-maleimide. In further embodiments the P-selectin-targeted liposomes comprise about 50 mole % hydrogenated soy L-α-phosphatidylcholine (HSPC), about 45 mole % cholesterol, about 3 mole % 1,2-distearoyl-sn-glycero-3-phosphoethanolamine-N-[(polyethylene glycol)2000] (DSPE-PEG2000) and about 2 mole % DSPE-PEG2000-maleimide. In yet further embodiments the P-selectin-targeted liposomes comprise 50 mole % hydrogenated soy L-α-phosphatidylcholine (HSPC), 45 mole % cholesterol, 3 mole % 1,2-distearoyl-sn-glycero-3-phosphoethanolamine-N-[(polyethylene glycol)2000] (DSPE-PEG2000) and 2 mole % DSPE-PEG2000-maleimide.

Provided is a composition comprising (i) P-selectin-targeted carriers comprising VEGF and (ii) MSCs. In some embodiments, the P-selectin-targeted carriers are P-selectin-targeted liposomes, P-selectin-targeted quantum dots or P-selectin-targeted biodegradable nanoparticles. In some embodiments, the P-selectin-targeted liposomes are immunoliposomes. In some embodiments the P-selectin-targeted liposomes comprise hydrogenated soy L-α-phosphatidylcholine, cholesterol, 1,2-distearoyl-sn-glycero-3-phosphoethanolamine-N-[(polyethylene glycol)2000] and DSPE-PEG2000-maleimide. In further embodiments the P-selectin-targeted liposomes comprise about 50 mole % hydrogenated soy L-α-phosphatidylcholine (HSPC), about 45 mole % cholesterol, about 3 mole % 1,2-distearoyl-sn-glycero-3-phosphoethanolamine-N-[(polyethylene glycol)2000] (DSPE-PEG2000) and about 2 mole % DSPE-PEG2000-maleimide. In yet further embodiments the P-selectin-targeted liposomes comprise 50 mole % hydrogenated soy L-α-phosphatidylcholine (HSPC), 45 mole % cholesterol, 3 mole % 1,2-distearoyl-sn-glycero-3-phosphoethanolamine-N-[(polyethylene glycol)2000] (DSPE-PEG2000) and 2 mole % DSPE-PEG2000-maleimide.

Also provided is a kit comprising, in a first compartment, a composition comprising P-selected-targeted carriers comprising VEGF, and, in a second compartment, a composition comprising MSCs. In some embodiments, the P-selectin-targeted carriers are P-selectin-targeted liposomes, P-selectin-targeted quantum dots or P-selectin-targeted biodegradable nanoparticles. In some embodiments, the P-selectin-targeted liposomes are immunoliposomes. In some embodiments the P-selectin-targeted liposomes comprise hydrogenated soy L-α-phosphatidylcholine, cholesterol, 1,2-distearoyl-sn-glycero-3-phosphoethanolamine-N-[(polyethylene glycol)2000] and DSPE-PEG2000-maleimide. In further embodiments the P-selectin-targeted liposomes comprise about 50 mole % hydrogenated soy L-α-phosphatidylcholine (HSPC), about 45 mole % cholesterol, about 3 mole % 1,2-distearoyl-sn-glycero-3-phosphoethanolamine-N-[(polyethylene glycol)2000] (DSPE-PEG2000) and about 2 mole % DSPE-PEG2000-maleimide. In yet further embodiments the P-selectin-targeted liposomes comprise 50 mole % hydrogenated soy L-α-phosphatidylcholine (HSPC), 45 mole % cholesterol, 3 mole % 1,2-distearoyl-sn-glycero-3-phosphoethanolamine-N-Kpolyethylene glycol)2000] (DSPE-PEG2000) and 2 mole % DSPE-PEG2000-maleimide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the net change in heart fractional shortening (“FS”) between the 1st and 4th week post-myocardial infarction (“post-MI”) in rats treated with either P-selectin-targeted immunoliposomes containing VEGF (1 ml of liposome/kg of animal weight at 10 mM lipid concentration) infused through tail vein immediately after induction of MI (“VEGF”), treated intramyocardially with MSCs one week post MI induction (“MSCs”), or with both P-selectin-targeted immunoliposomes containing VEGF and MSCs (“VEGF+MSCs”). “*” indicates a significant difference compared to “No Treatment” by ANOVA, p<0.001. “**” indicates a significant difference compared to all other treatments, by ANOVA, p<0.001.

FIGS. 2A-D illustrate collagen formation after MI in rats treated with either P-selectin-targeted immunoliposomes containing VEGF (1 ml of liposome/kg of animal weight at 10 mM lipid concentration) infused through tail vein immediately after induction of MI (“VEGF”), treated intramyocardially with MSCs one week post MI induction (“MSCs”), or treated with both P-selectin-targeted immunoliposomes containing VEGF and MSCs (“VEGF+MSCs”). Untreated (FIG. 2A), MSCs treated (FIG. 2B), targeted VEGF treated (FIG. 2C) and targeted VEGF+MSCs (FIG. 2D) heart sections were stained with Picrosirius Red. Images were taken using a circular polarizer. The scale bar represents 0.13 mm.

FIG. 3 illustrates the average intensity of collagen in untreated and treated MI rat heart sections based on Picrosirius Red staining. Rats were treated with either P-selectin-targeted immunoliposomes containing VEGF (1 ml of liposome/kg of animal weight at 10 mM lipid concentration) infused through tail vein immediately after induction of MI (“VEGF”), treated intramyocardially with MSCs one week post MI induction (“MSCs”), or with both P-selectin-targeted immunoliposomes containing VEGF and MSCs (“VEGF+MSCs”). “*” indicates significant difference compared to MI only group, by ANOVA p<0.001. “**” indicates a significant difference compared to all other treatments, by ANOVA p<0.05.

FIGS. 4A-E illustrate detection of anatomical vessels by CD31 staining in rats that were either normal (no MI) (FIG. 4A), untreated (FIG. 4B), treated intramyocardially with MSCs one week post MI induction (“MSCs”) (FIG. 4C), treated with P-selectin-targeted immunoliposomes containing VEGF (1 ml of liposome/kg of animal weight at 10 mM lipid concentration) infused through tail vein immediately after induction of MI (“VEGF”) (FIG. 4D) or treated with both P-selectin-targeted immunoliposomes containing VEGF and MSCs (“VEGF+MSCs”) (FIG. 4E). The CD31 staining was measured 4 weeks after MI induction. Blood vessels were stained, imaged and processed to remove background in order to count the number of blood vessels. The number of vessels was then normalized to the area to calculate vessel density. The scale bar represents 0.13 mm.

FIG. 5 illustrates the number of anatomical blood vessels, quantified from FIGS. 4A-E. The Y axis “Ratio” was calculated by normalizing the number of vessels in the infarcted myocardium of each heart sample to that in the normal myocardium of the same sample. Data are represented as “mean+standard error” (n=4 samples for each group). “*” indicates a significant difference compared to “No Treatment” group, by ANOVA, p<0.01.

FIG. 6 illustrates the number of anatomical blood vessels (n=3). CD31 staining was used to detect anatomical vessels in normal (no MI), untreated, MSCs treated, targeted VEGF treated, and targeted VEGF+MSCs treated rats, measured 4 weeks after MI induction. Blood vessels were stained, imaged and processed to remove background in order to count the number of blood vessels. The number of vessels was then normalized to the area to calculate vessel density.

FIGS. 7A-B illustrate MSCs in the infarcted myocardium as indicated by GFP fluorescence four weeks after grafting. Images were taken from MI transplanted with only MSCs (FIG. 7A) and from MI pre-treated with VEGF and then transplanted with MSCs (FIG. 7B). The scale bar represents 0.32 mm.

FIGS. 8A-D illustrate stem cells in MI, treated by MSCs+VEGF. Samples were stained with anti-cardiac troponin T antibody (FIG. 8A), anti-α-actinin (FIG. 8B), anti-vimentin (FIG. 8C) or anti-α-smooth muscle actin (FIG. 8D). Transplanted GFP-expressing MSCs are shown in green. Cy3-labeled antibodies are shown in red. The scale bar represents 0.1 mm.

### DEFINITIONS

As used in the specification and the appended claims, the singular forms “a,” “an” and “the” include plural referents unless the context clearly dictates otherwise.

The term “about” will be understood by persons of ordinary skill in the art and will vary to some extent depending on the context in which it is used. As used herein, “about” is meant to encompass variations of ±20% or ±10%, more preferably ±5%, even more preferably ±1%, and still more preferably ±0.1%.

As used herein, the term “antibody” refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies that may be used in the practice of the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, New York; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). The term “antibody” is intended to include antibody fragments that retain antigen-binding activity.

As used herein, the terms “treat” and “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms.

As used herein, “individual” (as in the subject of the treatment) includes human beings and non-human animals, including both mammals and non-mammals. Mammals include, for example, humans; non-human primates, e.g. apes and monkeys; cattle; horses; sheep; and goats. Non-mammals include, for example, fish and birds.

The expression “effective amount” in connection with the treatment of a patient suffering from myocardial infarction, refers to the amount of a component of the invention that when combined with the other component of the invention, aids in the recovery of heart function, or that maintains the disease in a state of complete or partial remission, or slows the progression of the disease.

As used herein “carriers” refers to compositions that may enclose, encapsulate, be conjugated with, or otherwise transport or carry a drug.

The phrase “liposomes” as used herein refers to unilamellar vesicles or multilamellar vesicles such as are described in U.S. Pat. No. 4,753,788, the contents of which are incorporated herein by reference. In some embodiments, the diameter of liposomes may vary from about 50 nm to about 250 nm. In preferred embodiments, the diameter of the liposomes is from about 100 nm to about 150 nm.

The phrase “unilamellar liposomes,” as used herein refers to spherical vesicles comprised of one lipid bilayer membrane which defines a single closed aqueous compartment. The bilayer membrane is composed of two layers of lipids; an inner layer and an outer layer (leaflet). The outer layer of the lipid molecules are oriented with their hydrophilic head portions towards the external aqueous environment and their hydrophobic tails pointed downward toward the interior of the liposome. The inner layer of the lipid lays directly beneath the outer layer, the lipids are oriented with their heads facing the aqueous interior of the liposome and their tails towards the tails of the outer layer of lipid.

The phrase “multilamellar liposomes,” as used herein refers to liposomes that are composed of more than one lipid bilayer membrane, which membranes define more than one closed aqueous compartment. The membranes are concentrically arranged so that the different membranes are separated by aqueous compartments, much like an onion.

The phrase “targeted carrier” as used herein refers to a carrier to which a targeting moiety is attached. The targeting moiety binds to a target on cells of the patient.

The phrase “targeted liposomes” as used herein refers to liposomes to which a targeting moiety is attached. The targeting moiety binds to a target on cells of the patient.

The phrase “immunoliposomes” as used herein refers to targeted liposomes wherein the targeting moiety is an antibody or an antibody fragment. Such antibody fragments include, but are not limited to, Fab and F(ab′)₂ fragments.

The phrase “targeted quantum dot” as used herein refers to a quantum dot to which a targeting moiety is attached. The targeting moiety binds to a target on cells of the patient.

The phrase “targeted biodegradable nanoparticle” as used herein refers to a biodegradable nanoparticle to which a targeting moiety is attached.

The phrase “stem cell” as used herein refers to a master cell that can differentiate indefinitely to form the specialized cells of tissues and organs. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells.

The phrase “mesenchymal stem cell” as used herein refers to a stem cell of mesenchymal origin. Mesenchymal stem cells are multipotent stromal cells that can differentiate into a variety of cell types, including osteoblasts, chondrocytes and adipocytes.

As envisioned in the present invention with respect to the disclosed compositions of matter and methods, in one aspect the embodiments of the invention comprise the components and/or steps disclosed therein. In another aspect, the embodiments of the invention consist essentially of the components and/or steps disclosed therein. In yet another aspect, the embodiments of the invention consist of the components and/or steps disclosed therein.

### DETAILED DESCRIPTION OF THE INVENTION

Myocardial infarction (MI) involves loss of blood flow in a specific region of the heart and the extent of the damage is dependent on location of the blockage and time after MI. Deep within the infarct area, cells devoid of an oxygen supply become apoptotic and/or terminally necrotic within hours and cannot be rescued even with reperfusion.

The methods and compositions of the invention may be used to treat a region of myocardial infarct because the carriers of the invention target P-selectin, a cell-adhesion molecule that is upregulated after MI, due to an upregulation of the inflammatory response. Without wishing to be bound by any theory, targeted delivery of VEGF to the infarcted myocardium border zone can lead to the growth of new blood vessels. The side-effects in distant tissue are minimized since the delivery of VEGF is targeted to the affected tissue. Infarcted regions that are treated with both MSCs and with P-selectin-targeted liposomes comprising VEGF exhibit an unexpected improvement that is more than additive when compared to infarcted regions that are treated with either component alone.

Provided are methods for treating myocardial infarction comprising administering to a subject in need of such a treatment an effective amount of a composition comprising a P-selectin-targeted carrier comprising VEGF and administering to the subject mesenchymal stem cells (MSCs). In some embodiments, the P-selectin-targeted carrier comprises a targeting moiety which binds P-selectin. In some embodiments, the targeting moiety is an antibody or an antibody fragment. Such antibody fragments include, but are not limited to, Fab and F(ab′)₂ fragments. In some embodiments, the antibody or an antibody fragment is attached via a linker to the carrier or carrier surface.

In some embodiments, the P-selectin-targeted carrier comprises P-selectin-targeted liposomes, P-selectin-targeted quatum dots or P-selectin-targeted biodegradable nanoparticles. In some embodiments, the comprises P-selectin-targeted liposomes are immunoliposomes. The targeting moiety attached to the immunoliposome carrier is an antibody or antibody fragment that binds P-selectin.

In some embodiments, the targeting moiety, e.g. antibody or an antibody fragment, is attached to the carrier via a linker. For example, where the carrier is a liposome, the targeting moiety may be attached to a linker connected to the distal end of PEG chains disposed in or on the liposome.

The carriers used in the practice of the present invention are compositions that may enclose, encapsulate, be conjugated with, or otherwise transport or carry a drug. In some embodiments, the carriers are particulate carriers. In some embodiments, carriers comprise liposomes, quantum dots or biodegradable nanoparticles. The carriers are targeted to P-selectin by an attached targeting moiety. The targeting moiety binds to P-selectin on cells of the patient.

### Liposomes

The liposomes utilized herein as P-selectin-targeted carriers may be either unilamellar or multilamellar in nature, the latter defining more than one closed aqueous compartment.

Suitable liposomes include those composed primarily of vesicle-forming lipids. Such a vesicle-forming lipid is one which can form spontaneously into bilayer vesicles in water. The lipid bilayer preferably contains at least a phospholipid as its main component.

The vesicle-forming lipids are preferably lipids having two hydrocarbon chains, typically acyl chains, and a head group, either polar or nonpolar. Synthetic vesicle-forming lipids and naturally-occurring vesicle-forming lipids include, for example, phospholipids, where the two hydrocarbon chains are typically between about 14-22 carbon atoms in length, and have varying degrees of unsaturation. The above-described lipids and phospholipids whose carbon chains have varying degrees of saturation can be obtained commercially or prepared according to published methods. Other suitable lipids include glycolipids, cerebrosides and sterols, such as cholesterol.

Exemplary phospholipids include glycerophospholipids such as phosphatidylcholine (lecithin), phosphatidylglycerol, phosphatidic acid, phosphatidylethanolamine, phosphatidylserine, and phosphatidylinocitol; sphingophospholipids such as sphingomyelin; natural or synthetic diphosphatidyl phospholipids such as cardiolipin, and derivatives thereof. The phospholipid may be hydrogenated by a method commonly used in the art (for example, hydrogenated soybean phosphatidyl choline). Preferred phospholipids are hydrogenated phospholipids such as hydrogenated soybean phosphatidyl choline (HSPC), e.g., hydrogenated soy L-α-phosphatidylcholine. The liposome may contain either single phospholipid or a plurality of phospholipids as its main membrane component.

The liposome may also contain a membrane component other than the main lipid component described above. For example, the liposome may be formed from a mixture of a phospholipid and a lipid other the phospholipid or a derivative of such lipid, capable of stable incorporation into lipid bilayers, to form the membrane of the liposome. Examples of such additional lipids include glyceroglycolipids, sphingoglycolipids, and sterols such as cholesterol.

The exterior of the lipid bilayer may be modified with a hydrophilic macromolecule. Examples include polyethylene glycol, polyglycerin, polypropylene glycol, polyvinyl alcohol, styrene-maleic anhydride alternating copolymer, divinyl ether-maleic anhydride alternating copolymer, polyvinylpyrrolidone, polyvinylmethylether, polyvinylmethyloxazoline, polyethyloxazoline, polyhydroxy-propyloxazoline, polyhydroxypropylmethacrylamide, polyumethacrylamide, polydimethylacrylamide, polyhydroxypropyl methacrylate, polyhydroxyethyl acrylate, hydroxymethylcellulose, hydroxyethylcellulose, polyaspartamide, and synthetic polyamino acid. The hydrophilic macromolecule can increase prolong the stability of the liposome in vivo. Preferred hydrophilic macromolecules are polyethylene glycol (PEG), polyglycerin (PG), and polypropylene glycol (PPG). The PEG may typically have a molecular weight of 500 to 10,000 Daltons, preferably 1,000 to 7,000 Daltons, and more preferably 2,000 to 4,000 Daltons.

The hydrophilic macromolecule moiety may be introduced into the liposome membrane by a lipid derivative of the hydrophilic macromolecule. In certain embodiments, the lipid portion of the lipid derivative of the hydrophilic macromolecule may comprise, for example, a phospholipid. The acyl chain included in the phospholipid may comprise, for example, a saturated fatty acid, for example a C₁₄-C₂₀, fatty acid. Exemplary of such acyl chains include dipalmitoyl, distearoyl, and palmitoyl stearoyl.

The selection of lipids is generally guided by considerations of liposome size and ease of liposome sizing, and lipid and water soluble drug release rates from the site of liposome delivery. The degree of saturation can be important since hydrogenated phospholipid components have greater stiffness than do unhydrogenated phospholipid components. This means that liposomes made with hydrogenated phospholipid components will be more rigid.

The phospholipid portion of the lipid derivative of the hydrophilic macromolecule may include a functional group which can react with the hydrophilic macromolecule to form the lipid derivative of the hydrophilic macromolecule. Examples of a phospholipid having a functional group reactive with the hydrophilic macromolecule include phospholipids comprising amino, hydroxyl and carboxy groups, such as phosphatidyl ethanolamine (amino group), phosphatidylglycerol (hydroxy group), and phosphatidylserine (carboxy group).

Particular lipid derivatives of the hydrophilic macromolecules may be formed by the bonding of a phospholipid; an additional lipid such as sterol; an aliphatic alcohol; an aliphatic amine; a glycerin fatty acid ester; and PEG, PG, or PPG, with PEG being preferred. One such preferred one PEG-derivatized phospholipid is 1,2-distearoyl-sn-glycero-3-phosphoethanolamine-N-[(polyethylene glycol) (DSPE-PEG). In one embodiment, the PEG has a molecular weight of about 2,000 Daltaons (DSPE-PEG2000). In preferred embodiments, the liposome comprises at least one PEG-derivatized phospholipid. Incorporation of PEG-derivatized lipids in liposomal membranes results in formation of sterically stabilized liposomes (“stealth liposomes”), possessing an extended long circulation time. See e.g., Lasic et al., Curr Opin Mol Ther 1:177-185 (1999); Lasic, Nature 380:561-562 (1996).

In some embodiments the P-selectin-targeted liposomes comprise hydrogenated soy L-α-phosphatidylcholine, cholesterol, 1,2-distearoyl-sn-glycero-3-phosphoethanolamine-N-[(polyethylene glycol)2000] and DSPE-PEG2000-maleimide. In further embodiments the P-selectin-targeted liposomes comprise about 50 mole % hydrogenated soy L-α-phosphatidylcholine (HSPC), about 45 mole % cholesterol, about 3 mole % 1,2-distearoyl-sn-glycero-3-phosphoethanolamine-N-[(polyethylene glycol)2000] (DSPE-PEG2000) and about 2 mole % DSPE-PEG2000-maleimide. In yet further embodiments the P-selectin-targeted liposomes comprise 50 mole % hydrogenated soy L-α-phosphatidylcholine (HSPC), 45 mole % cholesterol, 3 mole % 1,2-distearoyl-sn-glycero-3-phosphoethanolamine-N-[(polyethylene glycol)2000] (DSPE-PEG2000) and 2 mole % DSPE-PEG2000-maleimide.

Preparation of vesicle-forming lipids derivatized with hydrophilic polymers has been described, for example in U.S. Pat. No. 5,395,619. Preparation of liposomes including such derivatized lipids has also been described, where typically between 1-20 mole percent of such a derivatized lipid is included in the liposome formulation (see, for example, U.S. Pat. No. 5,013,556).

The liposomes may be formed according to well-known methods, which are summarized, for example, in U.S. Pat. No. 8,241,663. For example, to form liposomes, the component lipids are mixed. Then the lipids are dried under vacuum overnight. The resulting lipid film is rehydrated in 40° C. deionized water to form vesicles. The lipids are then extruded using, e.g., a Lipex™ extruder (Vancouver, BC, Canada) ten times with a 0.2 micron filter (Nucleopore) yielding a liposome diameter of 196±3 nm (Scott et al., FASEB J. 23(10): 3361-3367, 2009).

In some embodiments, the diameter of liposomes may vary from about 50 nm to about 250 nm. In preferred embodiments, the diameter of the liposomes is from about 100 nm to about 150 nm.

For targeting to P-selectin, the liposome exterior surface may be suitably formed or derivatized to contain a functional group comprising, or for attaching to, a P-selecting targeting moiety. Where the targeted liposome is an immunoliposome, the liposome-forming materials in one embodiment may include a component comprising a functional group capable of linking to a functional group of an antibody or antibody fragment, or to an appropriately modified functional group of an antibody or antibody fragment. Such antibody fragments include, but are not limited to, Fab and F(ab′)₂ fragments. In some embodiments, antibody or antibody fragment is attached to the distal end of PEG chains on the liposome surface to form immunoliposomes via a linker. In preferred embodiments, the linker is maleimide. In further preferred embodiments, the antibody or antibody fragment is thiolated with an agent that introduces thiol groups.

In some embodiments, the antibody or antibody fragment is covalently attached to the free distal end of a hydrophilic polymer chain, e.g., PEG, which is attached to or embedded in a vesicle-forming lipid. There are a wide variety of techniques for attaching a selected hydrophilic polymer to a selected lipid and activating the free, unattached end of the polymer for reaction with a selected ligand, in particular, PEG. See, e.g., Allen, et al., Biochemicia et Biophysica Acta, 1237:99-108 (1995); Zalipsky, Bioconjugate Chem., 4(4):296-299 (1993); Zalipsky, et al. FEBS Lett, 353:71-74 (1994); Zalipsky, et al., Bioconjugate Chemistry, 6(6):705-708 (1995); Zalipsky, in STEALTH LIPOSOMES (D. Lasic and F. Martin, Eds.) Chapter 9, CRC Press, Boca Raton, Fla. (1995)).

Generally, the hydrophilic polymer, e.g., PEG, chains may be functionalized to contain reactive groups suitable for coupling with, for example, sulfhydryls, amino groups, and aldehydes or ketones (typically derived from mild oxidation of carbohydrate portions of an antibody) present in a wide variety of ligands. Thus, in some embodiments, an antibody or antibody fragment is attached to the distal end of PEG chains on the liposome surface to form immunoliposomes via a linker. Examples of such PEG-terminal reactive groups include maleimide (for reaction with sulfhydryl groups), N-hydroxysuccinimide (NHS) or NHS-carbonate ester (for reaction with primary amines), hydrazide or hydrazine (for reaction with aldehydes or ketones), iodoacetyl (preferentially reactive with sulfhydryl groups) and dithiopyridine (thiol-reactive). Synthetic reaction schemes for activating PEG with such groups are set forth in U.S. Pat. Nos. 5,631,018, 5,527,528, 5,395,619, and the relevant sections describing synthetic reaction procedures are expressly incorporated herein by reference.

In certain embodiments, the immunoliposome comprises a PEG-terminal reactive maleimide, for coupling with a thiolated antibody/antibody fragment which will serve as the targeting moiety on the immunoliposome. Maleimide functionalization may be achieved by including in the vesicle-forming lipids an appropriately maleimide-functionalized lipid, such as 1,2-distearoyl-sn-glycero-3-phosphoethanolamine-N-[(polyethylene glycol)-maleimide, e.g., DSPE-PEG2000 maleimide.

In one embodiment, antibody is first thiolated with 2-iminothiolane (Sigma-Aldrich Corporation, St. Louis, Mo., USA) at pH 8.0. The introduced thiol groups are then coupled with maleimide groups on the DSPE-PEG2000 component of the liposomes at pH 6.5. Unconjugated antibodies are removed by, e.g., a HiTrap heparin column.

Targeted liposomes, such as immunoliposomes, may be prepared by allowing drug-loaded (i.e., loaded with VEGF) vesicles to react with the appropriate targeting ligand under appropriate conditions. To prepare immunoliposomes, the antibody used as the targeting ligand may be first modified to generate appropriate reactive groups on the antibody for covalent binding to liposome components. For example, the antibody is modified with succinimidyl acetylthioacetate (SATA) or 2-iminothiolane (Traut's reagent) at pH 8.0 to introduce thiol groups (Derksen et al., Biochimica et Biophysica Acta 814: 151-155, 1985). The thiolated antibody is then coupled with maleimide groups, such as a DSPE-PEG2000 component of the liposomes, at pH 6.5. In certain embodiments, the molar ratio of Ab-SH to maleimide is 1:40 using an Ab-SH concentration of 0.3 mg/ml. The reaction is carried out >12 hours at 4° C., is stopped and unconjugated antibody is separated from the liposomes by, e.g., ultracentrifugation for 1 hour at 30,000 rpm. Alternatively, the antibody-containing liposomes can be separated from free antibody by column chromatography using, e.g., a Sephadex CL-4B column pre-equilibrated with saline. Previous studies (Nallamothu et al., AAPS Pharm Sci Tech 7: E32, 2006; Pattillo et al., Pharm Res 22: 1117-1120, 2005) have shown that this results in a sufficient number of antibodies being attached to each liposomal particle to cause an attachment of the particle to an antigen. While a 1:40 ratio of Ab-SH to maleimide group is an acceptable ratio for antibody attachment, other ratios, such as ratios in the range of 1:10 to 1:100 may be utilized to optimize drug delivery.

While antibody attachment to drug-loaded liposomes is exemplified by resort to drug-loaded liposomes comprising DSPE-PEG2000 as the membrane component for antibody attachment, other membrane components may be substituted for DSPE-PEG2000 in providing functional groups for covalent attachment of antibodies and antibody fragments.

### Drug Loading of Immunoliposomes

VEGF loading of liposomes may be accomplished, for example, by a method in which the lipid layer constituting the liposome is hydrated with an aqueous solution containing the drug to thereby load the drug in the liposome (passive loading), or by a method in which an ion gradient is formed between the interior and the exterior of the liposome layer so that the drug permeates through the liposome layer according to the ion gradient to become loaded in the liposome (remote loading). See e.g., U.S. Pat. No. 5,192,549, and U.S. Pat. No. 5,316,771.

In one method, VEGF may be encapsulated into the hydrophilic interior of immunoliposomes by the freeze dry and rehydration method (Patillo et al., Pharm Res. 2005 July; 22(7)1117-20. Epub 2005 Jul. 22; Scott et al., Biotech. Bioeng. 2007 Mar. 1; 96(4):795-802). Immunoliposome-entrapped drug may be determined using Endogen® Human VEGF ELISA Kit from Pierce Biotechnology, Inc. Un-encapsulated drug may be separated from immunoliposomes by passing a diluted sample through a column such as a Sephadex G-50 column. The eluent containing immunoliposomes is then assayed for drug and phospholipids. Lipid content is estimated followed by a phosphate assay. Total drug incorporated is calculated by first rupturing the vesicles followed by the determination of free drug in solution using the VEGF ELISA Kit. Trapping efficiencies are then calculated as the drug/phospholipids ratio after separation of free drug divided by the drug/phospholipids ratio before separation.

### Quantum Dots

In certain embodiments, the carrier for VEGF comprises semiconductor nanocrystals, also known as quantum dots. The dots are fluorescent tags that may be associated with a drug, reagent or molecule. They may be conjugated to a P-selectin targeting moiety such as an antibody or antibody fragment, either directly or via a linker, to become targeted quantum dots that can bind to the antibody target (Gao et al., Nature Biotechnology. 2004; 22(8):969-976; Tan et al., Biomaterials. 2007, 28(8):1565-1571). Quantum dots may be prepared as described in U.S. Pat. No. 6,326,144, the contents of which are incorporated herein by reference. In some embodiments, the quantum dots have a diameter of from about 12 Å to about 150 Å.

When quantum dots are illuminated with a primary energy source, a secondary emission of energy occurs of a frequency that corresponds to the band gap of the semiconductor material used in the quantum dot. Many semiconductors that are constructed of elements from groups II-IV, III-V and IV of the periodic table have been prepared as quantum sized particles, exhibit quantum confinement effects in their physical properties, and can be used in the composition of the invention. Exemplary materials suitable for use as quantum dots include ZnS, ZnSe, ZnTe, CdS, CdSe, CdTe, GaN, GaP, GaAs, GaSb, InP, InAs, InSb, AlS, AlP, AlAs, AlSb, PbS, PbSe, Ge, and Si and ternary and quaternary mixtures thereof. The quantum dots may further include an overcoating layer of a semiconductor having a greater band gap.

### Biodegradable Nanoparticles

In certain embodiments, the carrier for VEGF comprises biodegradable nanoparticles. The nanoparticles may be conjugated to a P-selectin targeting moiety such as an antibody or antibody fragment, either directly or via a linker, to become P-selectin-targeted biodegradable nanoparticles. The biodegradable nanoparticles are complexed with VEGF. The biodegradable nanoparticles may be prepared using any method known in the art for preparing nanoparticles. Such methods include spray drying, emulsion-solvent evaporation, double emulsion and phase inversion. For methods for preparing biodegradable nanoparticles complexed with drugs, see U.S. Patent Application No. 2010/0303912 and U.S. Application No. 2004/0247683, the contents of which are incorporated herein by reference.

The naturally occurring polymer used to make the biodegradable nanoparticles usually has a high molecular weight, e.g., an average molecular weight of 25,000 or greater. In general, the naturally occurring polymer has a molecular weight of about 50,000 to about 1,000,000, and preferably about 75,000 to about 750,000. In some embodiments, the naturally occurring polymer has a molecular weight of about 100,000 to about 700,000. In some embodiments the biodegradable nanoparticles have a diameter of from about 10 nm to about 1000 nm. In some preferred embodiments, the biodegradable nanoparticles have a diameter of from about 50 nm to about 250 nm.

Some suitable naturally occurring polymers for preparing biodegradable nanoparticles include, but are not limited to, dermatan sulfate, chondroitin sulfate, keratin sulfate, heparin sulfate, dextran sulfate, and mixtures thereof. Examples of other biodegradable polymers include, but are not limited to, collagen, albumin, cellulose, gelatin, elastin and hyaluronic acid.

### MSCs

MSCs for use in the practice of the present invention may isolated and cultured for transplantation by well-known methods. Methods of isolating MSCs are described, for example, in U.S. Patent Application Nos. 2010/0226976, 2008/0038231 and 2007/0053888, which are incorporated herein by reference in their entirety.

MSCs are also referred to as “marrow stromal cells” or “multipotent stromal cells”. The MSCs utilized in the practice of the present invention may be of a xenogeneic or allogeneic source. They may be obtained from the patient, from a different person, or from a family member of the patient, for example. The MSCs may be obtained after matching of the alleles of each human leukocyte antigen (HLA) locus within the major histocompatibility complex (MHC) between the donor and the recipient of the MSCs.

MSCs can be obtained from a number of tissues including for example bone marrow, embryonic yolk sac, placenta, umbilical cord, fetal and adolescent skin, peripheral blood and other tissues. MSCs may be stored and frozen after collection and prior to use. MSCs may be expanded ex vivo either after collection or prior to administration to the patient.

MSCs may be induced to differentiate by the addition of agents such as growth factors, hormones or interleukins, as described in U.S. Application No. 2007/0053888 which is incorporated by reference herein in its entirety. Agents that can induce stem or progenitor cell differentiation are well known in the art and include, but are not limited to, Ca²⁺, EGF, α-FGF, β-FGF, PDGF, keratinocyte growth factor (KGF), TGF-β, cytokines (e.g., IL-1α, IL-1β, IFN-γ, TFN), retinoic acid, transferrin, hormones (e.g., androgen, estrogen, insulin, prolactin, triiodothyronine, hydrocortisone, dexamethasone), sodium butyrate, TPA, DMSO, NMF, DMF, matrix elements (e.g., collagen, laminin, heparin sulfate, Matrigel™), or combinations thereof. In certain embodiments, MSCs or progenitor cells are induced to differentiate into a particular cell type, according to methods well known in the art.

MSCs may be induced to proliferate, for example, by administration of erythropoietin, cytokines, lymphokines, interferons, colony stimulating factors (CSFs), interferons, chemokines, interleukins, recombinant human hematopoietic growth factors including ligands, stem cell factors, thrombopoeitin (Tpo), interleukins, and granulocyte colony-stimulating factor (G-CSF) or other growth factors.

MSCs may be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

In preferred embodiments, the MSCs are isolated from a human. In further preferred embodiments, the MSCs are isolated from bone marrow.

MSCs for administration according to the present invention may be grown in, for example, D-MEM media (low glucose) with GlutaMax™-I and are supplemented with 10% MSC-Qualified FBS, and 5 mg/ml Gentamicin. MSCs are passaged every 2-3 days using 0.025% TypLE Express Dissociation Reagent and are grown at 37° C. with 5% CO₂. All the materials for MSC culture may be obtained, for example, from Life Technologies (Grand Island, N.Y., USA).

### Administration of Therapy

Treatment for myocardial infarction according to the present invention is achieved by administering a combination of (i) P-selectin-targeted carriers comprising VEGF and (ii) MSCs. In one embodiment of the invention, the combination of P-selectin-targeted carriers and MSCs are co-formulated and used as part of a single composition that is administered intramyocardially.

Alternatively, according to other embodiments of the invention, the combination of P-selectin-targeted carriers containing VEGF and MSCs may be formulated and administered as two or more separate compositions, at least one of which comprises at least P-selectin targeted carriers containing VEGF and at least one of which comprises MSCs. The MSC composition is administered intramyocardially. The separate VEGF-carrier composition may be administered by the same or different routes as the MSC composition, administered at the same time or different times, and administered according to the same schedule or on different schedules, providing the dosing regimen is sufficient to bring about the desired effect. When the VEGF-carrier composition and MSCs are administered in serial fashion, it may prove practical to intercalate administration of the two compositions, wherein a time interval, for example a 0.1 to 48 hour period, separates administration of the two drugs.

Non-limiting routes of administration for the P-selectin targeted carriers containing VEGF include local intravenous and intramyocardial administration, for example intramyocardial injection. Other routes of injections are systemic intravenous injection, systemic intraarterial injection, and local intracoronary artery injection. In one embodiment, both the P-selectin-targeted carriers and the MSCs are delivered locally to the site of a cardiac lesion by intramyocardial injection. In some embodiments, the P-selectin-targeted carriers comprising VEGF are injected intravenously, and the MSCs are delivered locally to the site of a cardiac lesion by intramyocardial injection.

It will be appreciated that “administered” means the act of making a drug available to the patient such that a physiological effect is realized. Thus, contemplated within the scope of the present invention is the instillation of the P-selectin-targeted carriers or the MSCs or both in the body of the patient in a controlled or delayed release formulation, with systemic or local release of the active agents occurring at a later time and/or over a prolonged time interval.

A depot of a first agent may be administered to the patient and the therapy component comprising the other agent may be administered prior to, subsequent to, or during the systemic release of the first agent.

In one embodiment, MI patients are injected with an effective amount of a VEGF-carrying immunoliposome solution. In preferred embodiments, 1 ml/kg of a 10 mM immunoliposome solution with arhVEGF165 concentration of 200 ng/ml may be employed. VEGF165 at local doses of 1-10 ng/mL has been reported to stimulate HUVEC proliferation (BelAiba et al., Eur J Biochem 268: 4398-4407, 2001; Machens et al., J Surg Res 111: 136-142, 2003; Takeuchi et al., J Pharmacol Sci 103: 168-174, 2007).

MSCs are preferably administered within one week after MI. This may be accomplished by exposing the heart via thoracotomy incision. Cultured MSCs may be injected intramyocardially in, for instance, 4 locations from the base to the apex around the infarcted border of the heart. In preferred embodiments, 25 μl of MSCs are injected at each location.

According to one embodiment, liposome-free VEGF may be additionally administered to the subject undergoing combined MSC and VEGF P-selectin targeted carrier therapy. Accordingly, a recombinant human VEGF, e.g. VEGF165, may be administered intravenously (50 ng/kg/min) for 200 minutes on days 1, 4 and 7 post-MI for a total infusion dose of 30 μg/kg. VEGF165 at systemic doses of 10-50 μg/kg has been reported to stimulate angiogenesis (Hughes et al., Annals of Thoracic Surgery 77: 812-818, 2004; Sato et al., Journal of the American College of Cardiology 37: 616-623, 2001).

Noninvasive modality of echocardiography may be utilized in order to follow the morphologic, hemodynamic and functional changes seen during treatment. Transthoracic echocardiography (Phillips SONOS 5500; 5-12 MHz multi-frequency transducer) may be used to assess the changes in left ventricular geometry and function in animals after myocardial infarction and after receiving treatment. The treatment may be carried out for as long a period as necessary while monitoring these parameters. The treating physician will know how to increase, decrease or interrupt treatment based on patient response.

### Kits

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration. Also optionally included with such container(s) are instructions for carrying out the methods of the invention.

The instructional material may comprise a publication, a recording, a diagram, or any other medium of expression which can be used to communicate the usefulness of the method. The package insert may comprise text housed in any physical medium, e.g., paper, cardboard, film, or may be housed in an electronic medium such as a diskette, chip, memory stick or other electronic storage form. The instructional material of the kit of the invention may, for example, be affixed to a container which contains other contents of the kit, or be shipped together with a container which contains the kit. Alternatively, the instructional material may be shipped separately from the container with the intention that the instructional material and the contents of the kit be used cooperatively by the recipient.

The practice of the invention is illustrated by the following non-limiting examples.

### EXAMPLES

### Example 1

### Preparation of Immunoliposomes Conjugated to Anti-P-Selectin Monoclonal Antibody

The targeted drug delivery system was produced by a two-step process as described earlier (Scott et al., Biotechnol Bioeng. 2007; 96:795-802). First, VEGF (Genentech, San Francisco, Calif., USA) loaded long circulating liposomes were composed of 50% hydrogenated soy L-α-phosphatidylcholine (HSPC), 45% cholesterol, 3% 1,2-distearoyl-sn-glycero-3-phosphoethanolamine-N-[(polyethylene glycol)2000] (DSPE-PEG2000), and 2% DSPE-PEG-maleimide and were prepared by means of solvent evaporation and film formation. All lipids were obtained from Avanti Polar Lipids, Inc. (Alabaster, Ala., USA). Second, anti P-selectin monoclonal antibody (courtesy of Dr. Andrew Issekutz) was attached to the distal end of PEG chains on the liposome surface to form immunoliposomes. P-selectin was chosen as the target receptor because it is upregulated on the vasculature of infarct tissue. The antibody was first thiolated with 2-iminothiolane (Sigma-Aldrich Corporation, St. Louis, Mo., USA) at pH 8.0. The introduced thiol groups were then coupled with maleimide groups on the DSPE-PEG2000 component of the liposomes at pH 6.5. Unconjugated antibodies were removed by HiTrap heparin column (GE Healthcare Biosciences, Pittsburgh, Pa., USA).

Long circulating liposomes were composed of HSPC, cholesterol, and DSPE-PEG in a molar ratio 50:45:5. For preparation of long circulating liposomes with attached anti-P-selectin, a fraction of DSPE-PEG (2 mol %) was replaced by DSPE-PEG-maleimide functional lipid. Liposomes were prepared by extrusion. Briefly, lipids dissolved in chloroform were mixed in appropriate amounts, the solvent was evaporated by a stream of nitrogen and then the sample was placed in a lyophilizer overnight to thoroughly evaporate excess chloroform. Afterwards, the thin dry film of lipid was suspended in a buffer containing therapeutic drugs (20 mM Tris-HCl, 135 mM NaCl, pH=7.2 if no antibody was to be attached, or pH 6.0 when antibody was to be coupled) preheated at 50° C. and vortexed for 3 min. Then the suspension was extruded (Lipidex, Vancouver, Canada) 10 times at 50° C. through a membrane of 200 nm pore size (Patillo et al., Pharm Res. 2005 July; 22(7)1117-20. Epub 2005 Jul. 22).

### Example 2

### Rat MI model

Surgical MI was induced in 6-week-old male Sprague Dawley rats (Charles River Laboratories International Inc, Wilmington, Mass., USA) as described previously (Wang B et al., Am J Physiol Heart Circ Physiol. 2005; 289:H108-113). Briefly, rats were anesthetized with isoflurane, intubated and ventilated with a rodent ventilator (Euthanex Corporation, Allentown, Pa., USA). The left anterior descending artery was occluded with silk ligature. Evidence of MI was confirmed by S-T segment elevation and the appearance of Q wave on an electrocardiogram. After surgery, rats were randomly assigned into four experimental groups: I) No treatment (saline injection), n=5 rats; II) targeted delivery of VEGF treatment alone, n=7 rats; III) MSCs treatment alone, n=7 rats and IV) targeted delivery of VEGF+MSCs treatment, n=10 rats.

### MSCs Culture

Rat bone marrow-derived MSCs expressing GFP were cultured as described previously (Del Valle et al., Cancer Biol Ther. 2010; 9). In brief, bone marrow of adult rat was flushed out from femurs and tibias under sterile conditions and plated in culture flasks in α-MEM (Mediatech) supplemented with 20% fetal bovine serum (FBS, Atlanta Biologicals), 2 mM L-glutamine, 100 μg/ml penicillin/streptomycin, and 25 μg/ml amphotericin B (Mediatech). The MSCs were isolated by their adherence to tissue culture plate. The non-adherent components were discarded. After isolation, MSCs were cultured in α-MEM media with 10% FBS supplemented with 10 μg/ml of Fibroblast Growth Factor (Invitrogen) and 10 μg/ml of Epidermal Growth Factor (Invitrogen).

Injection of P-Selectin Immunoliposomes Comprising VEGF and MSCs into Rat MI Model

P-selectin immunoliposomes containing VEGF (1 ml of liposome/kg of animal weight at 10 mM lipid concentration) were infused into rats through tail vein immediately after induction of MI. The VEGF content was about 200 ng/ml of liposome. MSCs were injected intramyocardially one week post MI induction. Briefly, 100 μl of MSCs at a concentration of 10,000 cells/μl were equally injected to 4 different sites (25 μl each) around the MI area in order to achieve a homogenous stem cell distribution.

### Determination of Fractional Shortening Changes

Rat heart function was monitored by echocardiography before, 1 week and 4 weeks post MI surgery. Left ventricular percent fractional shortening (FS) data were used to determine the heart function. Left Ventricle FS is a standard measure of the pumping function of the heart. Since normal rat usually has a FS value of greater than 40%, rats with a FS value of less than 30% were considered having a fully developed MI at the 1 week post MI surgery time point. A one-sided paired t-test was performed for each treatment between 1 week and 4 weeks post MI surgery time point. Differences among the groups at the 4 weeks post MI time point were determined using one way ANOVA with SNK post-hoc correction.

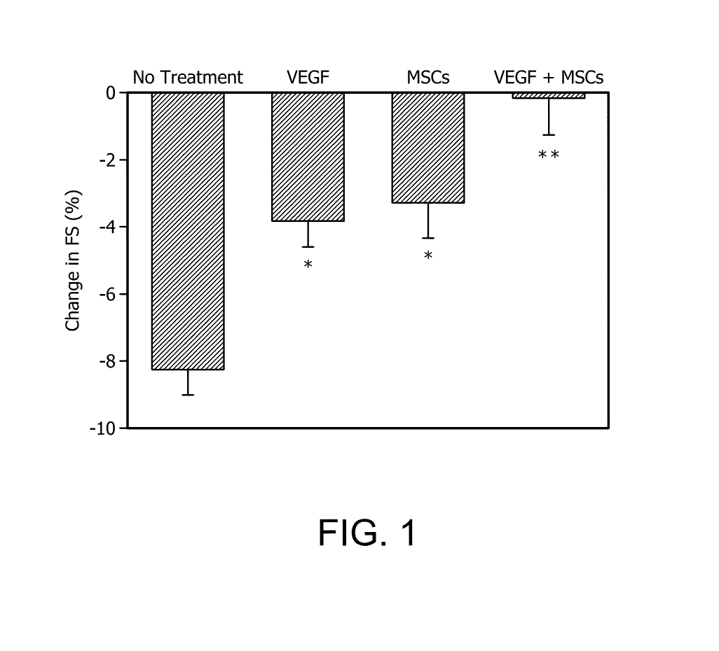

Analysis of FS revealed a significant loss in cardiac function in non-treated rats between 1 and 4 weeks post MI (FS: 18.7%±2.7% to 10.4%±3.4% by paired t-test, p<0.001). Either treatment with P-selectin-targeted immunoliposomes comprising VEGF or MSC treatment alone moderated this function loss (VEGF: 21.0%±5.2% to 17.2%+4.8, p<0.01; MSC: 20.1%+2.9% to 16.8%±5.0%, p<0.01). However, further loss in cardiac function was not observed in the VEGF+MSC treated rats. As shown in FIG. 1, the VEGF+MSC treatment group showed significantly better cardiac protective effect as compared to all other treatments (p<0.001 by ANOVA). The effect of VEGF+MSC was more than additive when compared to treatment with VEGF alone or with MSC alone.

### Example 3

### Histology and Immunohistochemistry

Four weeks after MI surgery animals were sacrificed and hearts were removed from rats and the ventricles were filled with OCT (Fisher Scientific Co., Houston, Tex., USA) to prevent chamber collapse. The hearts were then flash frozen in a dry ice/2-methylbutane (Fisher Scientific Co., Houston, Tex., USA) bath and stored at −80° C. Prior to staining, hearts were first sectioned to 9 μm slices at −20° C. using a cryostat (Leica CM3050 S, Buffalo Grove, Ill., USA) and then mounted on poly-L-lysine-coated glass slides (Superfrost plus, Fisher Scientific Co., Houston, Tex., USA) for staining and imaging.

The extent of tissue remodeling in the MI area was visalized using Gomori's Trichrome and Picrosirius red staining (both from Polysciences, Inc., Warrington, Pa., USA). The number of identified blood vessels was then counted by a built in function in ImagePro software (Media Cybernetics, Bethesda, Md., USA).

### Image Processing

A TE200 inverted microscope (Nikon Instruments Inc., Melville, N.Y., USA) was used to obtain images on stained sections. Images of a whole heart section were taken with the aid of a LEP MAC 5000 motorized stage (Ludl Electronic Products Ltd., Hawthorne, N.Y., USA) controlled by ImagePro.

### Determination of Morphological Changes and Tissue Remodeling in the MI Area.

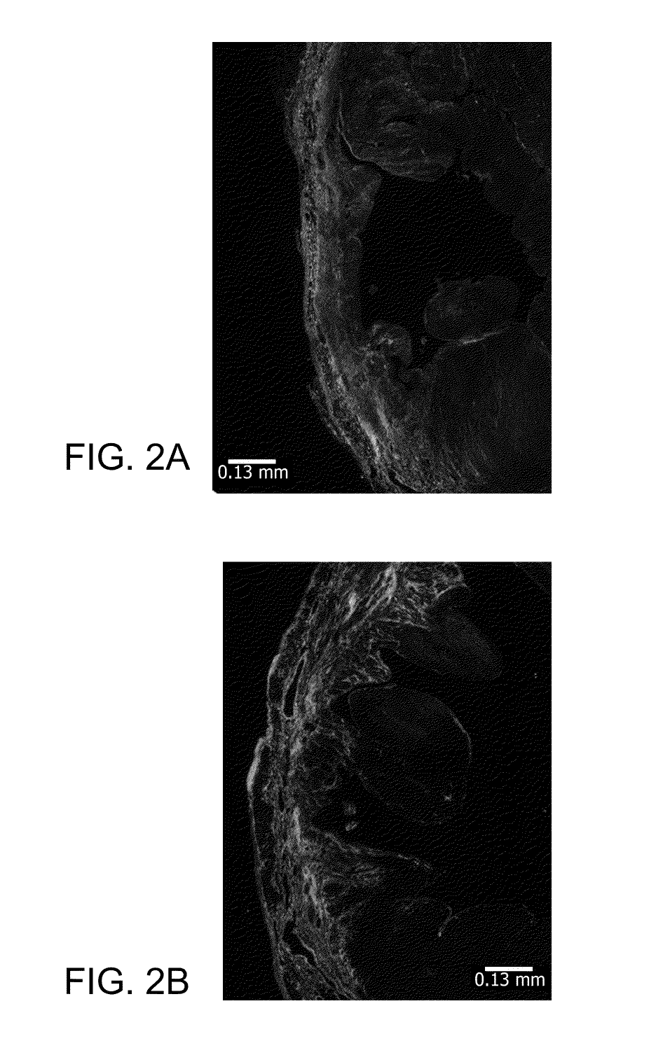

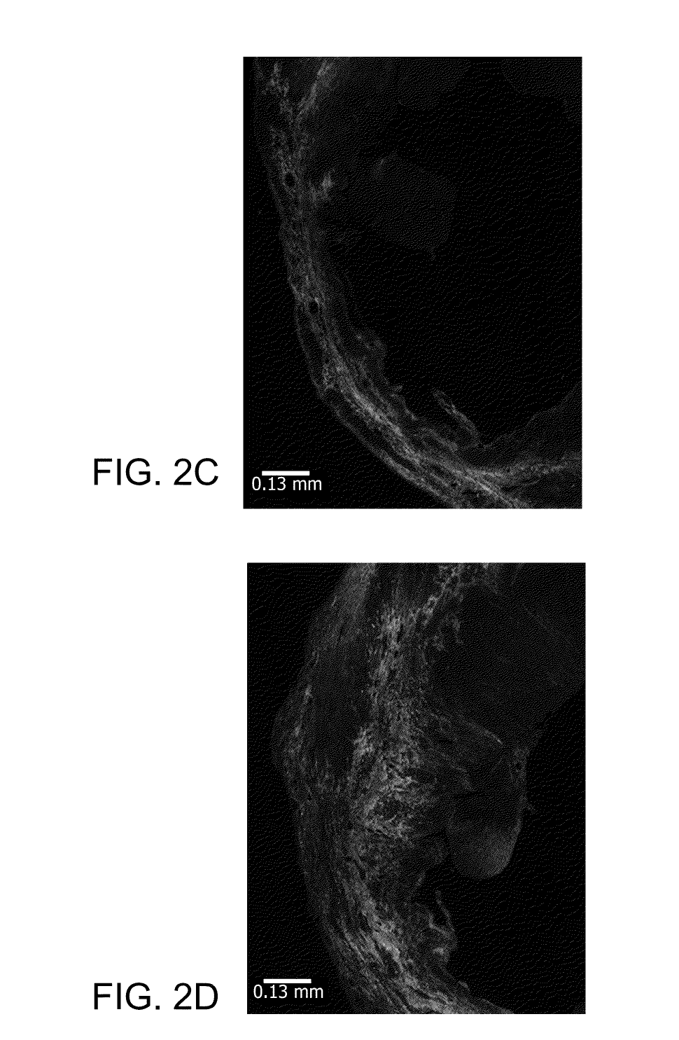

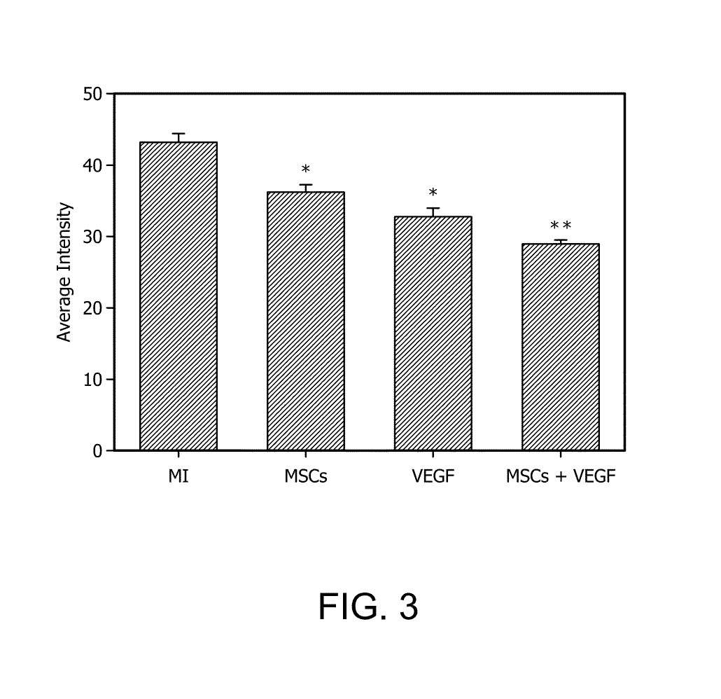

Picrosirius Red (FIGS. 2A-2D) staining was used to visualize collagen content in the MI area for non-treated and treated heart sections. When Sirius red in saturated picric acid selectively binds to fibrillar collagens (types I to V), it enhances the birefringence of fibrillar collagen. The specificity of Sirius red staining for fibrillar collagen enables sensitive quantitative measurements of collagen content to be performed in locally defined tissue areas. Quantification of collagen was based on the average intensity of red in the MI area (Rosano et al., Cardiovascular Engineering and Technology. 2012; 3:237-247). The results are shown in FIGS. 2A-2D: Untreated (FIG. 2A), MSCs treated (FIG. 2B), targeted VEGF treated (FIG. 2C) and targeted VEGF+MSCs (FIG. 2D) heart sections. Although the size of the infarction varies in each sample, quantitative analysis suggested that the percentage of collagen in the MI area decreased significantly with different treatments. As shown in FIG. 3, the collagen content decreased by 16%, 24% and 33% for MSCs alone, VEGF alone and MSCs+VEGF treatments, respectively, as compared to non-treated MI samples.

### Example 4

### Blood Vessel Regeneration in MI Area

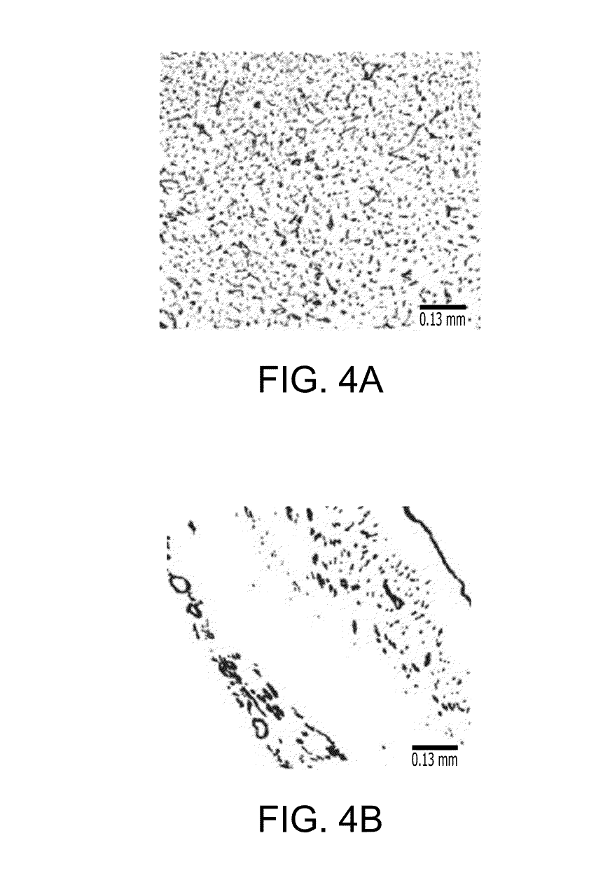

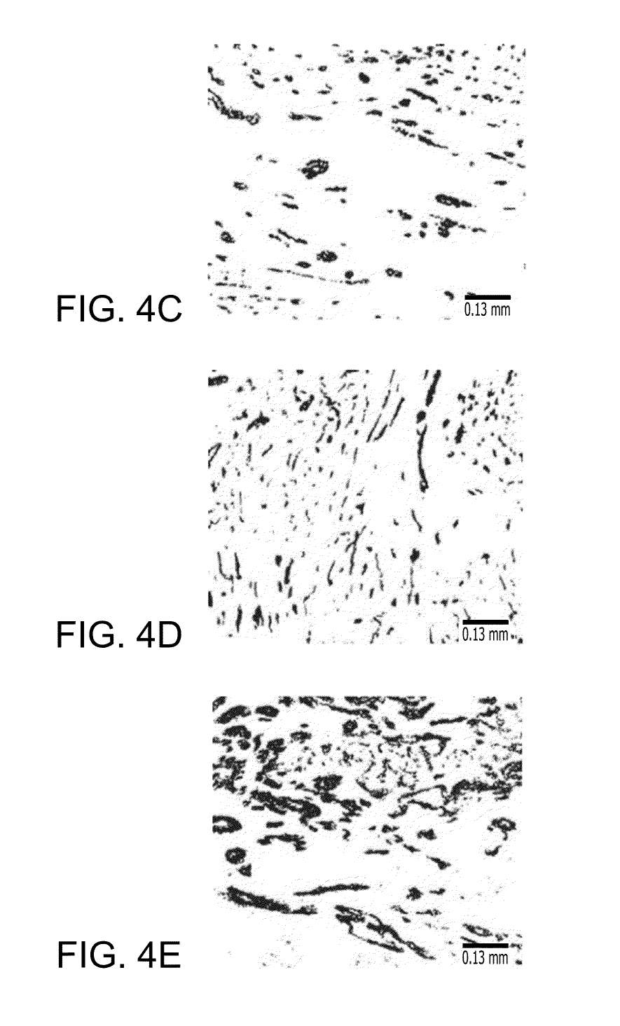

Anatomical blood vessel density was quantified by CD31 staining. Immunohistochemical staining for CD31 protein (BD Biosciences, Franklin Lakes, N.J., USA) was carried out as described in Example 3 for other markers. CD31 images were taken under bright field and enhanced using ImagePro to identify AEC-stained blood vessels. As shown in FIGS. 4A-4E, blood vessel density decreased significantly while the remaining blood vessels seem to be dilated in the untreated infarcted myocardium when measured 4 weeks after MI induction: (no MI) (FIG. 4A), untreated (FIG. 4B), treated intramyocardially with MSCs one week post MI induction (“MSCs”) (FIG. 4C).

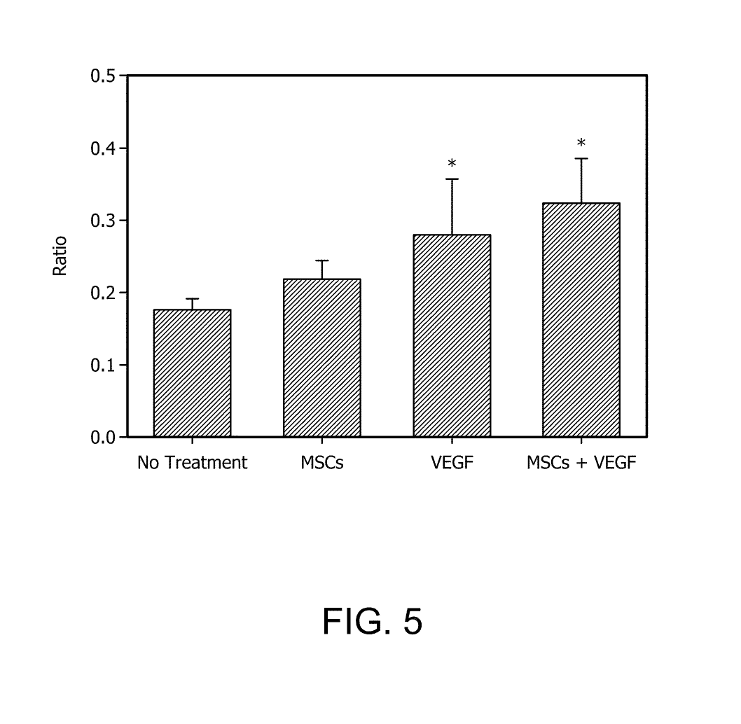

FIG. 5 illustrates the number of anatomical blood vessels, quantified from FIGS. 4A-E. As shown in FIG. 5 improvements in heart function were accompanied by a marked increase in vascular density observed in all three treatments groups. Compared to untreated MI group, stem cell treatment alone resulted in a slight but not significant increase in the number of anatomical blood vessels. VEGF treatment alone significantly promoted angiogenesis in the MI area. There was a significant increase in the number of anatomical blood vessels in VEGF+MSCs group indicating that combined VEGF+MSCs treatment was the most effective in promoting vessel growth in MI tissue.

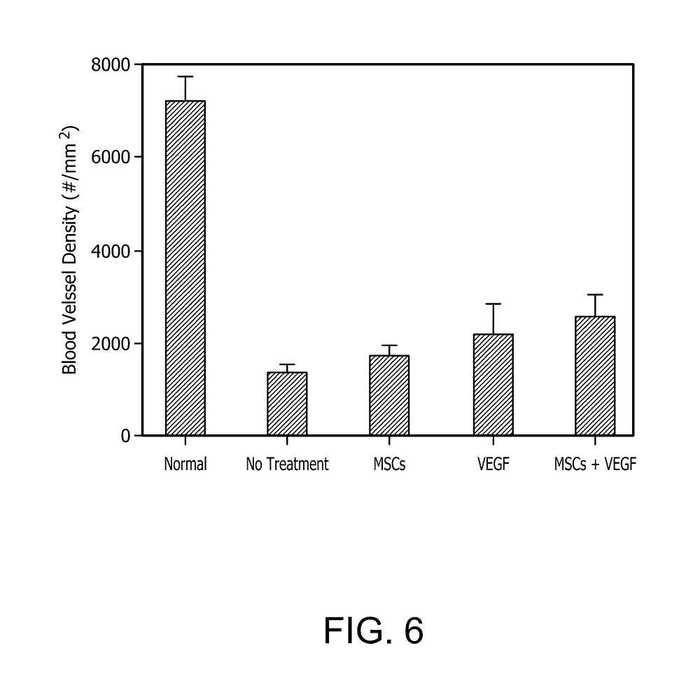

As shown in FIG. 6, compared to the untreated MI heart samples, stem cell treatment alone slightly increased the number of anatomical blood vessels, but both the VEGF treatment and the VEGF+MSCs combination therapy remarkably promoted angiogenesis in the MI area.

### Example 5

### MSCs Engraftment

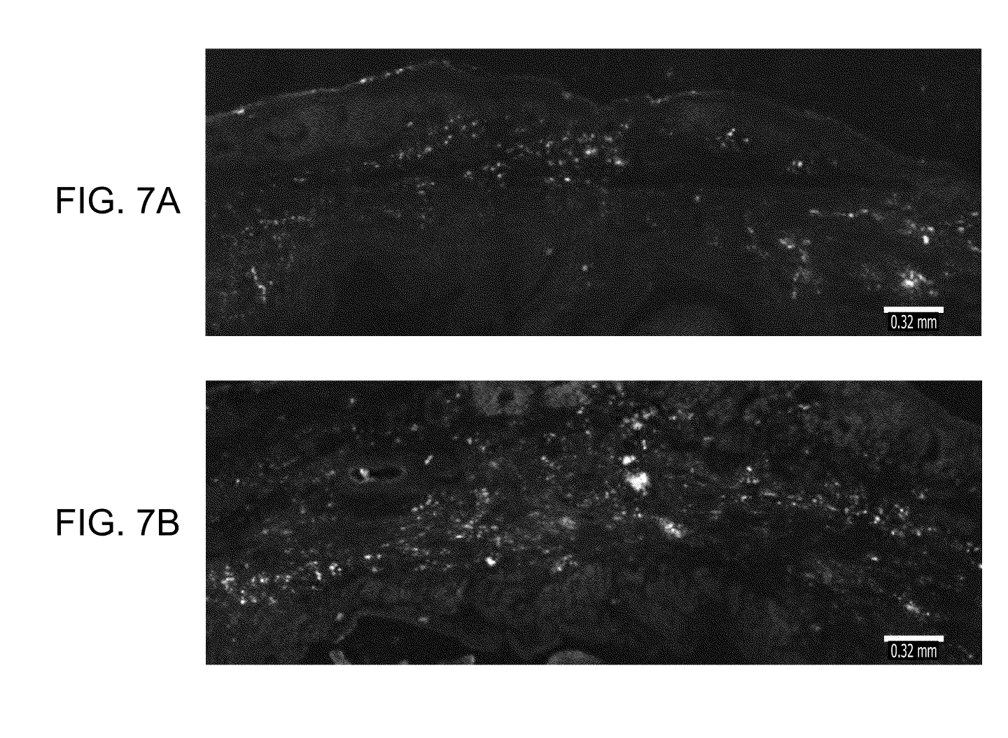

Total number and density of GFP expressing MSCs was determined from their fluorescence signature. As shown in FIGS. 7A (MSC transplantation only) and 7B (MSC transplantation+targeted VEGF pretreatment), targeted VEGF treatment greatly increased the total number and density of engrafted MSCs. Quantitative analysis (Table 1) indicated a 49.8% increase in total survived MSCs and 58.4% higher MSC density in VEGF+MSCs treated MIs. Data in Table 1 (n=4 samples) are represented as mean±standard deviation. “*” illustrates p<0.05. “**” illustrates p<0.01.

TABLE 1

| | | | Percentage |
|---|---|---|---|
| Treatment | MSCs Only | MSCs + VEGF | Increase |
| Number | 6147.25 ± 1763.54 | 9210.50 ± 1036.49 | 49.83% (*) |
| Density | 2179.49 ± 364.28 | 3543.35 ± 665.10 | 58.45% (**) |

### Example 6

### Determination of the Fate of MSCs

The phenotype of MSCs was determined by immunohistochemistry four weeks after cell transplantations. Identification of engrafted GFP expressing MSCs was carried out by using four different immunostainings (Thermo Fisher Scientific Inc., Rockford, Ill., USA). Monoclonal anti-cardiac troponin T antibody and monoclonal anti-α-actinin antibody were used to label cardiomyocytes; monoclonal anti-α-smooth muscle actin together with monoclonal anti-vimentin were used to identify newly formed blood vessels (Gabbiani et al. Proc Natl Acad Sci USA. 1981; 78:298-302; Santos et al., Zoolog Sci. 2012; 29:437-443; Juniantito et al., Exp Toxicol Pathol. 2012; 65(5): 567-577).

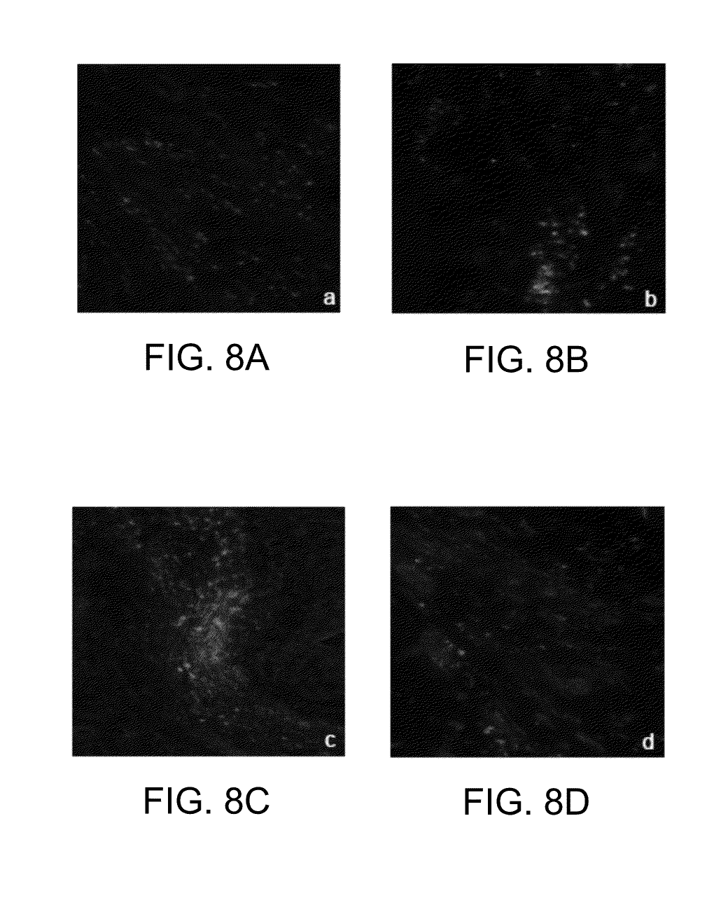

Anti-cardiac troponin T, anti-α-actinin, anti-α-smooth muscle actin and anti-vimentin staining were imaged using their corresponding fluorescence channels. The images were then superimposed with stem cell fluorescence images to find their viability and differentiation. For each heart section, the number of vessels in the normal area as well as infarct area (excluding border zone) were quantified and compared. The data are shown in FIGS. 8A-8D. Samples were stained with anti-cardiac troponin T antibody (FIG. 8A), anti-α-actinin (FIG. 8B), anti-vimentin (FIG. 8C) or anti-α-smooth muscle actin (FIG. 8D).

As shown in FIGS. 8A and 8B, both anti-cardiac troponin T and anti-α-actinin immunoreactivity are weak in GFP positive cells indicating that very few donor-derived cardiac myocytes are present in the MI area 4 weeks after the transplantation. The implanted cells are positive for vimentin and anti-smooth muscle actin, see FIGS. 8C and 8D, suggesting the formation of blood vessels.

The disclosures of each and every patent, patent application, publication and GenBank record cited herein are hereby incorporated herein by reference in their entirety.

One skilled in the art will readily appreciate that the present invention is well adapted to carry out the objects and obtain the ends and advantages mentioned, as well as those inherent therein. While the invention has been disclosed with reference to specific embodiments, it is apparent that other embodiments and variations of this invention may be devised by others skilled in the art without departing from the true spirit and scope used in the practice of the invention. The appended claims are intended to be construed to include all such embodiments and equivalent variations.

What is claimed is:

1. A method for treating myocardial infarction comprising administering to a subject in need of such a treatment an effective amount of a composition comprising P-selectin-targeted carriers comprising VEGF, and intramyocardially administering to said subject an effective amount of mesenchymal stem cells (MSCs).

2. The method of claim 1 wherein said P-selectin-targeted carriers are P-selectin-targeted liposomes, P-selectin-targeted quantum dots or P-selectin-targeted biodegradable nanoparticles.

3. The method of claim 2 wherein said P-selectin-targeted liposomes are immunoliposomes.

4. The method of claim 1 wherein said P-selectin-targeted liposomes comprise hydrogenated soy L-α-phosphatidylcholine, cholesterol, 1,2-distearoyl-sn-glycero-3-phosphoethanolamine-N-[(polyethylene glycol)2000] and DSPE-PEG2000-maleimide.

5. The method of claim 4 wherein said P-selectin-targeted liposomes comprise 50 mole % hydrogenated soy L-α-phosphatidylcholine (HSPC), 45 mole % cholesterol, 3 mole % 1,2-distearoyl-sn-glycero-3-phosphoethanolamine-N-[(polyethylene glycol)2000] (DSPE-PEG2000) and 2 mole % DSPE-PEG2000-maleimide.

6. A composition comprising (i) P-selectin-targeted carriers comprising VEGF and (ii) MSCs.

7. The composition of claim 6 wherein said P-selectin-targeted carriers are P-selectin-targeted liposomes, P-selectin-targeted quantum dots or P-selectin-targeted biodegradable nanoparticles.

8. The composition of claim 7 wherein said P-selectin-targeted liposomes are immunoliposomes.

9. The composition of claim 7 wherein said P-selectin-targeted liposomes comprise hydrogenated soy L-α-phosphatidylcholine, cholesterol, 1,2-distearoyl-sn-glycero-3-phosphoethanolamine-N-[(polyethylene glycol)2000] and DSPE-PEG2000-maleimide.

10. The composition of claim 9 wherein said P-selectin-targeted liposomes comprise 50 mole % hydrogenated soy L-α-phosphatidylcholine (HSPC), 45 mole % cholesterol, 3 mole % 1,2-distearoyl-sn-glycero-3-phosphoethanolamine-N-[(polyethylene glycol)2000] (DSPE-PEG2000) and 2 mole % DSPE-PEG2000-maleimide.

11. A kit comprising, in a first compartment, a composition comprising P-selectin-targeted carriers comprising VEGF, and, in a second compartment, a composition comprising MSCs.

12. The kit of claim 11 wherein said P-selectin-targeted carriers are P-selectin-targeted liposomes, P-selectin-targeted quantum dots or P-selectin-targeted biodegradable nanoparticles.

13. The kit of claim 12 wherein said P-selectin-targeted liposomes are immunoliposomes.

14. The kit of claim 12 wherein said P-selectin-targeted liposomes comprise hydrogenated soy L-α-phosphatidylcholine, cholesterol, 1,2-distearoyl-sn-glycero-3-phosphoethanolamine-N-[(polyethylene glycol)2000] and DSPE-PEG2000-maleimide.

15. The kit of claim 14 wherein said P-selectin-targeted liposomes comprise 50 mole % hydrogenated soy L-α-phosphatidylcholine (HSPC), 45 mole % cholesterol, 3 mole % 1,2-distearoyl-sn-glycero-3-phosphoethanolamine-N-[(polyethylene glycol)2000] (DSPE-PEG2000) and 2 mole % DSPE-PEG2000-maleimide.
